(19) United States
(12) Patent Application Publication
(10) Pub. No.: US 2014/0275751 A1
(43) Pub. Date: Sep. 18, 2014

(54) COLLAGEN-BASED OPHTHALMIC INTERFACE FOR LASER OPHTHALMIC SURGERY

(71) Applicant: Abbott Medical Optics Inc., Santa Ana, CA (US)

(72) Inventors: Robert G. Heitel, Laguna Beach, CA (US); Teresita (Tessie) Smith, Lake Forest, CA (US); Mark Evan Steen, Santa Ana, CA (US)

(21) Appl. No.: 14/190,777

(22) Filed: Feb. 26, 2014

Related U.S. Application Data
(60) Provisional application No. 61/788,917, filed on Mar. 15, 2013.

(51) Int. Cl. A61F 9/009; A61B 3/113; A61B 3/14; A61B 1/32; A61B 19/00

(52) U.S. Cl. CPC A61F 9/009; A61B 1/32; A61B 19/54; A61B 3/145; A61B 3/113
USPC 600/37; 128/845; 600/236

(57) ABSTRACT

Embodiments of a collagen-based ophthalmic interface for reducing patient eye movement are disclosed. In one embodiment, an ophthalmic interface serving as a partial barrier between a patient's eye and a surgical laser system includes an annular-shaped collagen-based shield configured to overlay the anterior surface of the eye. The shield is applied directly to the eye with an eyelid speculum, and reduces eye movement by adding friction to the surface of the eye. In another embodiment, a collagen-based material coats an attachment ring of a conical interface for coupling a patient's eye to a surgical laser system. The collagen-based coat glues the surface of the eye to the conical interface, reducing eye movement, and simultaneously eliminating the need for vacuum suction to hold the device in place. The gap in the middle of the annular-shaped collagen-coated attachment ring is filled with a liquid whose refractive index matches that of the cornea.

### CROSS-REFERENCES TO RELATED APPLICATIONS

This application claims priority to U.S. provisional application No. 61/788,917 filed on Mar. 15, 2013, the entire contents of which are incorporated herein by reference.

### TECHNICAL FIELD

Embodiments of the present invention generally relate to laser eye surgery, and more particularly to collagen-based ophthalmic interfaces for stabilizing a patient's eye movement in relation to a laser beam during ophthalmic surgery.

### BACKGROUND

Eye surgery is now commonplace with some patients pursuing it as an elective procedure to avoid using contact lenses or glasses and others pursuing it to correct adverse conditions such as cataracts. Moreover, with recent developments in laser technology, laser surgery has become the technique of choice for ophthalmic procedures. Laser eye surgery typically uses different types of laser beams, such as ultraviolet lasers, infrared lasers, and near-infrared, ultra-short pulsed lasers, for various procedures and indications.

A surgical laser beam is preferred over manual tools like microkeratomes as it can be focused accurately on extremely small amounts of ocular tissue, thereby enhancing precision and reliability. For example, in the commonly-known LASIK (Laser Assisted In Situ Keratomileusis) procedure, an ultra-short pulsed laser is used to cut a corneal flap to expose the corneal stroma for photoablation with an excimer laser. Ultra-short pulsed lasers emit radiation with pulse durations as short as 10 femtoseconds and as long as 3 nanoseconds, and a wavelength between 300 nm and 3000 nm. Besides cutting corneal flaps, ultra-short pulsed lasers are used to perform cataract-related surgical procedures, including capsulorhexis, capsulotomy, as well as softening and/or breaking of the cataractous lens.

Laser eye surgery is performed while the patient is in a reclined position but awake, meaning that the patient's eyes are moving during the procedure. As would be expected, patient eye movement relative to the laser beam's focal point can undermine the laser's accuracy and precision, and may even result in permanent tissue damage. Hence, various devices and mechanisms are conventionally used to stabilize, reduce, and/or eliminate patient eye movement, which in turn, improves safety and surgical outcome.

Among other things, visual fixation targets, eye trackers, and/or eye stabilizing ophthalmic patient interfaces are used to address eye movement. Visual fixation techniques essentially involve having the patient focus on a visual fixation target produced by a light emitting diode (LED), which is optically placed in front of or above the patient within his or her line of vision. Watching the fixation light helps the patient maintain a steady gaze, thus reducing random eye movement. Exemplary systems and methods for visual fixation are described in U.S. Pat. No. 6,004,313 and U.S. Pat. No. 6,406,473, issued to Shimmick et al., and U.S. Pat. No. 6,793,654, issued to Lemberg, which are incorporated here by reference. While visual fixation techniques work to some extent, the patient bears a significant burden of minimizing relative motion. Furthermore, the technique relies on the patient's conscious responses to the fixation target, and as such, is less tolerant of any significant gross autonomous reflex motion that could occur, for example, when a patient is startled.

Eye tracking techniques, on the other hand, do not impose as much burden on the patient. Eye tracking systems and devices monitor the position of a selected feature of the eye and provide the laser system with real time signals about any displacement in the position as a result of movement during surgery. Then, as necessary, the surgical laser system uses the signals to adjust or re-position the focal point of the laser beam before making an incision. Some examples of eye tracking systems and techniques are disclosed in U.S. Pat. No. 6,299,307, issued to Oltean et al., which is incorporated here by reference. Eye tracking systems are inordinately expensive as a second, independent optical path is usually provided between a patient's eye and the surgical laser to accommodate the eye tracking device. Further expense and complexity is also added as the eye tracker requires additional software that must be integrated into the surgical laser system. Moreover, to ensure accuracy and precision, the trajectory of the laser beam's focus must be corrected in real time, which is difficult as some involuntary eye movements are too rapid or erratic for the system to effectively track and offset. As such, inherent latency in an eye tracker and its interactions with the overall laser system may lengthen procedure times and/or adversely affect surgical outcomes.

Another technique for reducing eye movement uses a stabilization device such as an ophthalmic patient interface apparatus, which physically engages the anterior surface of a patient's eye. This device effectively eliminates eye movement. In addition, some ophthalmic patient interfaces can be used to align the eye and the surgical laser system. Generally, eye stabilization devices include either a rigid or a fluid ophthalmic patient interface, and a metal or rigid plastic conical adapter and an annular attachment ring. The large end of the conical adapter serves as a laser fixation mount while the small end of the conical adapter serves as a patient fixation mount. The attachment ring at the small end of the conical adapter is coupled with either a rigid contact lens or a fluid interface that is configured to overlay the anterior surface of a patient's eye. Typically, the attachment ring is further coupled with an annular skirt. The skirt is formed with a groove defining a suction channel between the skirt and the anterior surface of the eye. A vacuum source in communication with the channel is selectively activated to create a partial vacuum in the channel, which helps attach the eye to the ophthalmic patient interface device. The stabilization device may be disposable, thus preserving surgical sterility. Examples of ophthalmic patient interface devices used to stabilize the eye are described in U.S. Pat. No. 6,863,667, issued to Webb et al., U.S. Pat. No. D462,442 issued to Webb, U.S. Pat. No. 6,623,476, issued to Juhasz et al., and co-pending U.S. patent application Ser. No. 13/230,590, which are incorporated here by reference. While these devices effectively restrain eye movement, they have other challenges. A common complaint is that the mechanical pressure or vacuum suction used to attach the interfacing device to the eye causes discomfort and may contribute to post-operative pain and hemorrhaging. Another complaint is that patient discomfort and corneal wrinkling are exacerbated when the interfacing device uses a rigid contact lens to applanate or flatten the cornea as part of the surgical procedure.

In view of these challenges, there is a need for devices and methods that ensure patient comfort and safety as well as effectively restrain, reduce, and/or compensate for eye movement during laser ophthalmic surgery.

### SUMMARY OF THE INVENTION

Accordingly, embodiments of the present invention are directed to devices and methods for providing a collagen-based corneal interface that restrains and/or reduces eye movement during ophthalmic surgery and enhances eye-tracking, stabilization, and alignment techniques, thereby substantially obviating one or more problems due to limitations and disadvantages of the related art.

To achieve these objectives and other advantages, an embodiment of this invention provides an ophthalmic interface serving as a partial barrier between a patient's eye and a surgical laser system, comprising at least one annular-shaped shield configured to overlay the anterior surface of the eye, wherein the at least one annular-shaped shield is composed of a collagen-based material. In this embodiment, the collagen-based shield is applied directly to the anterior surface of the eye with an eyelid speculum or retractor whose blunt-ended arms are inserted underneath the upper and lower eyelids of a patient's eye to hold the lids apart to prevent the patient from blinking during surgery. The collagen-based shield reduces random and saccadic eye movement by adding friction, drag, or viscous effect to the anterior surface of the eye. Since the collagen-based shield has an annular shape, a central portion of the eye is open to receive the laser beam, while the collagen-based shield acts as an adhesive and glues down the rest of the eyeball.

In another embodiment, a collagen-based material coats an attachment ring of a conventional, cone-shaped, ophthalmic patient interface for coupling a patient's eye to a surgical laser system. In this embodiment, the collagen-based coat serves as an adhesive or glue to affix the conical device onto the anterior surface of the eye, thus effectively reducing eye movement while simultaneously eliminating the need for a vacuum suction mechanism to hold the device in place. The orifice or gap in the center of the collagen-coated ring may be filled with a fluid such that the annular collagen-based coat serves as a dam around the corneal fluid interface. In certain embodiments, the refractive index of the fluid may be matched to the refractive index of the cornea. Suitable fluids to form the interface include a balanced salt solution (BSS), ophthalmic viscoelastic devices, dextran-containing solutions, and/or combinations thereof.

In certain embodiments, the collagen-based ophthalmic interface may further contain reference marks or grids that can be captured by a video camera or followed by an eye-tracking device used in conjunction with the surgical laser system. For example, any relative movement between the eye and the reference marks or grid can be tracked by the eye tracker and/or captured by the video camera, thus enhancing the tracking capabilities of eye-tracker-guided surgical laser ophthalmic systems.

This summary and the following detailed description are merely exemplary, illustrative, and explanatory, and are not intended to limit, but to provide further explanation of the invention as claimed. Additional features and advantages of the invention will be set forth in the descriptions that follow, and in part will be apparent from the description, or may be learned by practice of the invention. The objectives and other advantages of the invention will be realized and attained by the structure particularly pointed out in the written description, claims and the appended drawings.

### BRIEF DESCRIPTION OF THE DRAWINGS

Understanding this invention will be facilitated by the following detailed description of the preferred embodiments considered in conjunction with the accompanying drawings, in which like numerals refer to like parts. Note, however, that the drawings are not drawn to scale.

FIG. 1 illustrates a diagram of an eye.

FIG. 2 illustrates an eyelid speculum used to deliver a collagen-based ophthalmic interface to the anterior surface of a patient's eye according to an embodiment of this invention.

FIG. 3 illustrates an example of reference marks or grid on a collagen-based shield according to an embodiment of this invention.

FIG. 4 illustrates a collagen-based shield according to an embodiment of this invention.

FIG. 5 illustrates an ophthalmic patient interface device having an attachment ring coated with a collagen-based material according to an embodiment of this invention.

FIG. 6 is a flow diagram illustrating a process according to an embodiment of this invention.

### DETAILED DESCRIPTION OF THE PREFERRED EMBODIMENTS

The drawings and related descriptions of the embodiments have been simplified to illustrate elements that are relevant for a clear understanding of these embodiments, while eliminating various other elements found in conventional collagen shields, ophthalmic patient interfaces, and in laser eye surgical systems. Those of ordinary skill in the art may thus recognize that other elements and/or steps are desirable and/or required in implementing the embodiments that are claimed and described. But, because those other elements and steps are well known in the art, and because they do not necessarily facilitate a better understanding of the embodiments, they are not discussed. This disclosure is directed to all applicable variations, modifications, changes, and implementations known to those skilled in the art. As such, the following detailed descriptions are merely illustrative and exemplary in nature and are not intended to limit the embodiments of the subject matter or the uses of such embodiments. As used in this application, the terms “exemplary” and “illustrative” mean “serving as an example, instance, or illustration.” Any implementation described as exemplary or illustrative is not meant to be construed as preferred or advantageous over other implementations. Further, there is no intention to be bound by any expressed or implied theory presented in the preceding background of the invention, brief summary, or the following detailed description.

Embodiments of this invention are directed to a collagen-based ophthalmic interface serving as a partial barrier between a patient's eye and a surgical laser system, wherein the interface is designed to reduce eye movement during surgery. FIG. 1 shows a cartoon diagram of an eye 10 with anatomical features, such as the cornea 14, the iris 26, the natural lens 16, the capsular bag 20, ciliary muscles 22, zonules 24, and the retina 12. Laser-eye surgery is typically performed on the cornea 14 to treat certain refractive conditions such as myopia, hyperopia, astigmatism, and the like. Surgical lasers are also used for cataract-related procedures, such as capsulotomy and capsulorhexis, where a capsular bag 20 is incised to gain access to a cataractous lens 16, which must be treated and/or removed to prevent blindness.

As mentioned earlier, laser eye surgery is performed while the patient is awake, so the patient's eyes are moving during the procedure. Patient eye movement relative to the focal point of the laser beam is evidently an issue as it can undermine the laser's accuracy and precision, which in turn may adversely affect the surgical outcome. Various different techniques using visual fixation targets, eye tracking, and/or ophthalmic patient interfaces are conventionally used to reduce, prevent, or compensate for random and saccadic eye movement during ophthalmic surgery. Yet, each technique often has its own challenge. Embodiments of the collagen-based ophthalmic interface may be used either by themselves, or together with other techniques, systems and devices to restrain, reduce, and/or compensate for patient eye movement.

In one embodiment, a collagen-based ophthalmic interface is applied directly to the anterior surface of an eye using an eyelid speculum or retractor. As shown in FIG. 2, an eyelid speculum 21 is often used to hold a person's eyelids 19 open for eye surgery, treatment, examination, or the like. The eyelid speculum 21 typically has two elongated blunt-ended arms which are inserted underneath the upper and the lower eyelids 19 to keep them apart, thus preventing the patient from blinking during the procedure. While the eyelid speculum 21 is stationary, the eyeball may still move. As shown, a portion of the annular-shaped, collagen-based ophthalmic interface 17 applied directly underneath the eyelids 19 with the speculum 21 may overlap portions of the sclera 15 of the eye, forming a partial barrier between the anterior surface of the eye and the atmosphere. The collagen-based interface 17 slows down random and saccadic eye movement by adding friction or viscous effect to the anterior surface of the eye. It also acts as an adhesive or glue, causing portions of the surface of the eye, such as the area beneath the eyelids, to adhere to the stationary eyelid speculum 21. This embodiment of the collagen-based ophthalmic interface may either be used on its own or in conjunction with other methods, such as visual fixation and/or eye tracking.

Among other materials, the collagen-based ophthalmic interface may be composed of a gelatin, a glycosaminoglycan, such as a chondroitin sulfate, and a carboxymethyl cellulose. The interface's biodegradability may be adjusted by varying the numbers of cross-linkers and/or varying the amounts of glycosaminoglycan and carboxymethyl cellulose. Hence, in some embodiments, the annular-shaped, collagen-based ophthalmic interface may be designed to remain on the eye for extended periods of time, such as throughout the operative phase of the procedure. In these embodiments, the collagen-based material may biodegrade or dissolve over time, be washed out with a solution, or be manually removed. In other embodiments, the collagen-based ophthalmic interface may be designed to dissolve within a shorter period. In some cases, the collagen-based ophthalmic interface may be applied to the eye as dressing. Further, in embodiments where the collagen-based interface dissolves quickly, additional collagen-based shields may be applied, i.e. the eye may be dressed with the collagen-based shields as often as necessary.

Because of its optical clarity, the collagen-based ophthalmic interface may be marked with reference marks or a grid that can be captured by a video camera and displayed on a video monitor used in conjunction with the surgical laser system. Alternately, an eye-tracking device could monitor and track the reference marks or grid and register any relative movement between the marks and the eye during surgery. FIG. 3 illustrates an example of reference marks 23 made on a collagen-based ophthalmic interface 28 contacting the anterior surface of a patient's eye 10 according to an embodiment of this invention. The reference marks may comprise various patterns, such as dots, lines, and the like, and may form a grid around the central portion of the eye 10 where the laser beam is delivered during surgery. The surgeon may place the reference marks 23 manually with a pen or a marker. Alternatively, a laser beam, which is operating at an energy level well-below that necessary for photodisruption could be used to place the reference marks 23 on the collagen-based ophthalmic interface 28 during the operative procedure.

For example, in one embodiment, a visual fixation device (not shown) could be used along with a collagen-based ophthalmic interface 28 overlaying the anterior surface of a patient's eye 10 to help reduce the patient's eye movement during surgery. When the patient's eye is fixated on a visual target, such as a light produced by the visual fixation device, a reference alignment is established between the eye's visual axis and that of the laser beam. When the patient's eye is initially in reference alignment at the beginning of surgery, the laser beam can be used to place marks 23 on the annular-shaped, collagen-based ophthalmic interface overlaying the anterior surface of the patient's eye. Hence, a known relationship between the marks 23 and the eye's visual axis would be established, which could be tracked by a video camera over the course of the surgical procedure.

FIG. 4 illustrates another embodiment in which a surgeon may manually place marks on the collagen-based ophthalmic shield overlying the anterior surface of a patient's eye during the pre-operative phase to identify distinguishing features, such as a particular blood vessel 25 on the sclera 27, a pattern on the iris 29, and the like. These marks may be particularly useful when transitioning the patient from the pre-operative diagnostic phase to the operative phase. Specifically, during the pre-operative phase, the patient sits in an upright position while his or her eyes are measured to assess the extent of abnormalities, such as refractive errors. Examples of ophthalmic diagnostic devices used for these measurements include the Abbott WaveScan WaveFront™ System and the Abbott iDesign Advanced WaveScan Studio aberrometer, which use a Shack-Hartmann wavefront sensor to quantify aberrations in a patient's eye. Although the measurements are made while the patient is upright, laser eye surgery is performed while the patient is lying down. This change in position—(upright to reclined)—causes the patient's eyes to rotate slightly, so treatment plans based on eye measurements in the upright position may not be exact when applied to the patient in a reclined position.

To resolve the treatment's precision limitations caused by ocular rotation, a collagen-based ophthalmic interface may be applied to the anterior surface of the patient's eye during the pre-operative phase according to an embodiment of the invention shown in FIG. 4. As such, while the surgeon is measuring the patient's eye and developing an appropriate treatment plan, he or she can place reference marks on the collagen-based ophthalmic shield to identify specific distinguishing features of the eye, such as a blood vessel 25 on the sclera 27, or a pattern on the iris 29. Because the marks would remain on the collagen-based ophthalmic interface throughout the surgical phase, they would allow the surgeon to monitor and to account for any ocular rotation that may have occurred due to the patient moving from an upright position to a reclined one.

As shown in FIG. 5, another embodiment provides an ophthalmic interface 41 for coupling a patient's eye to a surgical laser system, where the interface includes an attachment ring coated with a collagen-based material 57 configured to overlay the anterior surface of a patient's eye 10, a lens cone 51, and a containment chamber 59 configured to receive a liquid 55. The collagen-based coat 57 serves as an adhesive or glue to attach the attachment ring to the anterior surface of the eye 10. The lens cone 51 defines a first plane surface 45 configured to couple to a delivery tip of the surgical laser. An apex ring is coupled to the first plane surface, wherein the apex ring includes a distal end 47. The cone further includes a first receptacle 49 configured to receive the collagen-based material coated attachment ring 57 and a central cavity 43 configured to receive the lens cone. A containment chamber 59 configured to receive a liquid 55 is coupled to the lens cone 51 on the top end, and further coupled to the attachment ring 57 on the bottom end. Essentially, because the attachment ring coated with the collagen-based material 57 is annular in shape, an orifice or gap exists in the center portion when the attachment ring overlays the anterior surface of the eye 10. This gap may be filled with a fluid or liquid 55 such that the collagen-based ring forms a dam around the liquid in contact with the cornea 14. In this embodiment, the collagen-based ophthalmic interface reduces eye movement by gluing the eye 10 to the stationary conical device 41. Further, because the collagen-based ophthalmic interface 57 glues the conical device to the anterior surface of the eye, it eliminates the need for a vacuum mechanism to hold the device in place. As such, it effectively reduces eye movement while ensuring patient comfort and safety.

Various fluids may be used for the liquid interface 55 that is in contact with the cornea 14. In some embodiments, the liquid 55 may comprise a fluid or solution whose refractive index matches the refractive index of the cornea, which is approximately 1.39. Index matching reduces optical aberrations that may be introduced as the incident laser beam travels through the liquid to the eye for the treatment procedure. Suitable solutions and fluids for the liquid interface 55 include balanced salt solutions (BSS), ophthalmic viscoelastic devices, dextran-containing solutions, and/or combinations thereof. But, because solutions with high osmolality may cause dry eye syndrome or other discomfort, the osmolality of the liquid should not exceed 600 mOsm.

In some embodiments, the collagen-based ophthalmic interface may further include pharmaceutical agents and other therapeutically active substances, including topical drugs for ophthalmic indications, antivirals, antibiotics, steroidal and non-steroidal anti-inflammatory agents, mydiatrics, growth factors, anesthetics, analgesics, and the like, which can all be incorporated into the collagen-based shield 28 shown in FIG. 3, or into the attachment ring coated with collagen-based material 57 shown in FIG. 5. As such, the collagen-based ophthalmic interface serves as a therapeutic delivery vessel to deliver topical drugs and other therapeutics to the anterior surface of the eye.

FIG. 6 depicts a flow chart illustrating a process for reducing a patient's eye movement during surgery according to an embodiment of this invention. As shown, the process comprises forming an ophthalmic interface using a collagen-based material, and delivering the ophthalmic interface to an anterior surface of a patient's eye. The ophthalmic interface may be formed as a mixture, a coating, a gel, an annular-shaped shield, or any other composition or structure formable with collagen-based materials with the consistency, adhesiveness, and design required by the particular formation. As described earlier, the collagen-based material may include a gelatin, a glycosaminoglycan, such as a chondroitin sulfate, and a carboxymethyl cellulose. The interface's biodegradability may be adjusted by varying the numbers of cross-linkers and/or varying the amounts of glycosaminoglycan and carboxymethyl cellulose. As described in other parts of this application, in some embodiments, the collagen-based ophthalmic interface may be delivered to an anterior surface of a patient's eye directly using an eyelid speculum used to hold the eyelids apart during surgery. In other embodiments, the interface may be applied with a brush as dressing, and/or injected so as to overlay the anterior surface of the eye. In another embodiment, the collagen-based ophthalmic interface comprises a coating composition that is used to coat an attachment ring of a conical patient interface. The attachment ring coated with the collagen-based ophthalmic interface is configured to overlay an anterior surface of a patient's eye.

Although embodiments of this invention are described and pictured in an exemplary form with a certain degree of particularity, describing the best mode contemplated of carrying out the invention, and of the manner and process of making and using it, those skilled in the art will understand that various modifications, alternative constructions, changes, and variations can be made in the ophthalmic interface and method without departing from the spirit or scope of the invention. Thus, it is intended that this invention cover all modifications, alternative constructions, changes, variations, as well as the combinations and arrangements of parts, structures, and steps that come within the spirit and scope of the invention as generally expressed by the following claims and their equivalents.

What is claimed is:

1. An ophthalmic interface serving as a partial barrier between a patient's eye and a surgical laser system, comprising:
at least one annular-shaped shield configured to overlay the anterior surface of the eye, wherein the at least one shield is composed of a collagen-based material.

2. The ophthalmic interface of claim 1, wherein the at least one shield is applied directly to the anterior surface of the eye with an eyelid speculum used to hold the eyelids apart.

3. The ophthalmic interface of claim 1, wherein the at least one shield further comprises reference marks that can be captured by a video camera or an eye-tracking device.

4. The ophthalmic interface of claim 3, wherein the reference marks comprise a grid.

5. An interface for coupling a patient's eye to a surgical laser system, the interface comprising:
an attachment ring coated with a collagen-based material configured to overlay the anterior surface of the eye, the collagen-based material adhering to the eye and the attachment ring;
a lens cone defining a first plane surface configured to couple to a delivery tip of the surgical laser, the lens cone having:
an apex ring coupled to the first plane surface, the apex ring comprising a distal end;
a first receptacle configured to receive the attachment ring; and
a central cavity configured to receive the lens cone; and
a containment chamber configured to receive a liquid, the chamber further configured to couple with the lens cone and the attachment ring.

6. A method of stabilizing a patient's eye movement during ophthalmic surgery comprising:
forming an annular-shaped shield with a collagen-based material;
delivering the shield to a patient's eye such that the shield overlays an anterior surface of the eye.

7. The method of claim 6 wherein the delivering comprises inserting the shield underneath the upper and the lower eyelids of the patient's eye using an eyelid speculum.

8. A method of stabilizing a patient's eye movement during ophthalmic surgery comprising:
forming a coat with a collagen-based material;
coating an attachment ring of an ophthalmic patient interface with said the collagen-based coat;
delivering the attachment ring to the patient's eye such that the attachment ring overlays an anterior surface of the eye.

9. The method of claim 8 further comprising:
filling a gap in the central portion of the attachment ring overlaying an anterior surface of the eye with a liquid.
